(19) United States
(12) Patent Application Publication
(10) Pub. No.: US 2012/0192627 A1
(43) Pub. Date: Aug. 2, 2012

(54) Methods for Detecting and Monitoring the Activity of Energized Water and Other Liquids Useful for Enhancing the Alternative Cellular Energy Pathway in the Prevention and Therapy of Diseases

(76) Inventor: William John Martin, South Pasadena, CA (US)

(21) Appl. No.: 13/016,948

(22) Filed: Jan. 28, 2011

(51) Int. Cl. G01N 33/00

(52) U.S. Cl. 73/61.71

(57) ABSTRACT

A method for assaying an intrinsic energy property of water and other liquids is disclosed, as well as a means for detecting water energizing environmental energy fields. The method is based on the dynamic nature of dissolving neutral red dye particles added to the liquid and the light induced motion of remaining un-dissolved neutral red dye particles. The intrinsic energy activity of the liquid is related to the alternative cellular energy (ACE) pathway, which can function in the prevention and therapy of various illnesses in plants, animals and humans.

### CROSS REFERENCE TO RELATED APPLICATIONS

### Co-Pending Patent Application

Methods for Detection of Ultraviolet Light Reactive Alternative Cellular Energy Pigments (ACE-pigments) William John Martin Submitted Dec. 24, 2007. Publication number 20090163831
Method of assessing and of activating the alternative cellular energy (ACE) pathway in the Therapy of Diseases. William John Martin Submitted Submitted Jan. 16, 2008. Publication number 20090181467
Enerceutical mediated activation of the alternative cellular energy (ACE) pathway in the therapy of diseases. Submitted May 8, 2008. Publication number 20090280193
Enerceutical activation of the alternative cellular energy (ACE) pathway in therapy of diseases. Submitted Feb. 11, 2009. Publication number 20090202442
Method of using the body's alternative cellular energy pigments (ACE-pigments) in the therapy of diseases Submitted Feb. 20, 2009. Publication number 20100215763
Urine as a source of alternative cellular energy pigments (ACE-pigments) in the assessment and therapy of diseases. Submitted Mar. 5, 2009. Publication number 20100196297
Diagnostic value of systemic ACE pathway activation in the detection by fluorescence of localized pathological lesions. Submitted Jul. 26, 2010. Publication number 20100291000
Enerceutical mediated activation of the alternative cellular energy (ACE) pathway in the therapy of diseases. Submitted Jul. 2010.
Energy Charged Liquids to Enhance Enerceutical Activation of the Alternative Cellular Energy (ACE) Pathway in the Therapy of Diseases. Submitted Dec. 17, 2010. application Ser. No. 12/972,344
Energy Charged Alcoholic Beverages for Enhancing the Alternative Cellular Energy Pathway in the Prevention and Therapy of Diseases. Submitted January 2011. Application number

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not applicable.

### BACKGROUND OF THE INVENTION

In recently submitted co-pending patent applications, which are incorporated by reference herein, I disclosed that the passage of electrolytically derived Water Gas (otherwise known as Brown's Gas) into absolute alcohol greatly enhances the ability of the alcohol to react with neutral red dye and with alternative cellular energy (ACE) pigments (defined in the co-pending patent applications). When a small number (usually around 10-20) of neutral red dye particles (obtained directly from a commercially available neutral red dye powder e.g. Dudley Corp. N.J.), are gently sprinkled onto a plastic dish, which is partially filled with Water Gas “charged” alcohol; and the solution is illuminated with visible and/or UV light, there is rapid and vigorous dissolving of most of the neutral red dye, which occurs in a directional manner forming long, narrow red streams of dissolved neutral red. Equally impressive, fine particles, breaking away from the larger particles of neutral red dye, which remain un-dissolved in the alcohol solution, undergo continuing to-and-fro movements, with apparent attractive forces rapidly alternating with repulsive forces. The linearity of the dissolving neutral red and the dynamic movements of un-dissolved particles occur in regular (non-charged) absolute alcohol, but are greatly heightened by Water Gas charging of the alcohol. The use of charged alcohol also significantly enhances the intensity of the orange fluorescence of the neutral red solution, when compared with dye dissolved in non-charged alcohol. As the alcohol (both charged and non-charged) finally evaporates, the precipitating neutral red dye assumes attractive, banded circular patterns, as if being influenced by an interactive energy field. The patterns are more striking using charged alcohol. The charged alcohol also shows greater interaction than does non-charged alcohol with ACE pigments collected from the saliva and/or perspiration of virus infected patients. When used therapeutically, the combination of neutral red with charged alcohol is decidedly more effective in enhancing the ACE pathway in energy deficient individuals, when it was compared with benefits obtained using untreated (non-charged) alcohol.

The linear dissolving pattern of neutral red dye observed in alcohol solutions, was also observed in various alcoholic beverages, especially when they were charged with Water Gas. This extraordinary pattern is not ordinarily seen when neutral red dye is added to distilled water in similar plastic dishes. Instead, slowly dissolving neutral red particles become gradually surrounded by the dissolved dye in essentially concentric discs of red dye. An exception is seen with some plastic drinking glasses and some plastic covered paper cups. In these containers, there is an obvious attraction between the plastic material and the neutral red particles, leading to radial movements of the particles towards the interface between the surface of the water and the plastic container. (These types of containers are, therefore, unsuitable for used in the experiments described in this application). I have commonly used small (1.5″) square individual polycarbonate dishes and various multi-well tissue culture dishes. The dishes are generally observed using a low power, dissecting microscope with spacing between the light and the dish to help reduce heat transfer. In some experiments light emitting diode (LED) light was used to exclude heat, as opposed to light, as the cause of particle movements.

An important finding was observed when neutral red dye particles, are gently sprinkled (scattered) onto a plastic dish of distilled water, and the dish is placed in close proximity to a dish of charged alcohol with moving neutral red dye particles. Instead of the particles in the water remaining essentially stationary and only slowly dissolving, several of the particles will begin to move throughout the water. Moreover, if additional neutral red dye is added, the fresh particles will show more linear dissolving patterns with movements of the remaining un-dissolved particles. The energy transference effect is best seen when the dishes are in direct contact or comprise separate wells within the same multi-well dish. Physical contact is not absolutely necessary, however, and the effect has been seen between separated dishes. I, thereby, discovered that the altered dissolving pattern of neutral red dye in ordinary water potentially provided a simple assay for detecting a radiating energy coming from the well containing the charged alcohol and neutral red particles. The present application discloses another useful application of the basic “neutral red dye particle dissolving pattern and subsequent motions of the remaining un-dissolved particles” assay system. For convenience, this assay will be subsequently referred to as the “NR Kinetic Assay.” The new application is the assessment of the activity of either natural waters or water processed or stored in various ways, in their capacity to transfer kinetic energy to neutral red dye particles. This assay is being employed as a surrogate marker for the potential capacity of water, or other liquids, to effectively activate the ACE pathway of humans, animals and plants in the prevention and therapy of diseases.

Many individuals have claimed health enhancing and plant growth promoting activities of certain forms of water. Some of the claims relate to where the water is collected, implying possible geological influences on the water. Numerous methods have been employed to convert ordinary water into a more “energized” form. These methods include the use of a vortex to spin water; magnets to align water molecules; electromagnetic energies, including ultraviolet, visible light, infrared and radio wave transmissions, to impart specific resonance to the water; electrolysis of the water; and the addition of various components. Among the added components to potentially “activate” water are minerals, particularly magnesium and chlorides; humic and fulvic acids; zeolites; gases, including hydrogen and Water Gas; and undisclosed “proprietary” materials. Short of performing rigorous clinical trials, health claims of the benefits of the various water processing methods, are difficult to substantiate. Agricultural studies can be more objective but are rarely performed with adequate controls of possible confounding variables. Moreover, with some of the water products, the claims relate to what has been added to the water, e.g. minerals, rather than to an intrinsic property of the water. Measurements such as pH, Redox (ORP), surface tension, conductivity, supposed cluster size as assessed by nuclear magnetic resonance (NMR), etc., have not been shown to effectively discriminate between supposedly highly beneficial water from ordinary unprocessed water. There is, therefore, a need for a more meaningful assay of water activity, which can reasonably relate to its health enhancing and plant growth promoting activities. The NR Kinetic Assay is disclosed as a simple screening method to assess both naturally occurring and processed water samples for their potential to provide therapeutic benefits, presumably through the ACE pathway.

### BRIEF SUMMARY OF THE INVENTION

A procedure is disclosed that uses neutral red dye to assess an energy property of water and other liquids. The procedure is based upon the distinction between i) slowly dissolving stationary particles forming concentric areas of dissolved dyes, with any remaining un-dissolved dye, essentially remaining motionless and ii) more rapidly dissolving particles leading to a long linear single streams of red dye coming from individual particles, with considerable movements, which includes to-and-fro motions, of un-dissolved particles, which can also form into temporary small aggregates. With certain provisions, discussed later, the extent to which the more dynamic neutral red kinetic pattern occurs, is reasonably assumed to be a measure of the activity of the liquid to enhance the ACE pathway of a plant, animal and human. This correlation is strengthened when the dynamic activity occurring with neutral red dye added to an energized liquid can impart a discernable measure of this property to neutral red dye suspended in ordinary water. The NR kinetic assay can be applied to both naturally occurring liquids, including water, and to selection of processing methods to energize liquids for use in the prevention and therapy of diseases.

### BRIEF DESCRIPTION OF THE DRAWINGS

Not Applicable and none included

### DETAILED DESCRIPTION OF THE INVENTION

The NR Kinetic Assay has previously been employed in the direct assessment of various kinds of processed liquids, including absolute alcohol and alcoholic beverages. For instance, it was also used to demonstrate the marked effect of simply bubbling Water Gas through absolute alcohol on the ability of the “charged” alcohol to react with neutral red dye. Not only was there more of a direct effect on neutral red added to the Water Gas charged alcohol, such as more intense fluorescence, but the light illuminated charged alcohol solution containing neutral red dye, was clearly able to distantly induce movements of un-dissolved neutral red dye particles in water. In this assay, the light illuminated neutral red dye containing charged alcohol was placed in one of the wells of a tissue culture plate. Adjacent wells contained water plus stationary un-dissolved neutral red dye particles. These wells were observed microscopically to see whether any of the un-dissolved neutral red particles would begin to move, which they did beginning approximately 30-60 seconds later. An additional observation on the adjacent neutral red dye in water wells included the occasional formation of numerous gas bubbles, which might well represent the formation of Water Gas. The energy transference type of assay consistently provided positive results, which were not seen using multiple wells in other culture dishes, which simply had water plus neutral red solutions.

I was, particularly, interested in knowing whether the enhanced activity in these assays correlated with the efficacy of using the charged alcohol plus neutral red dye in a therapeutic procedure aimed at activating the ACE pathway. (Details of the procedure are provided in some of the co-pending patent applications.) Essentially, the neutral red dye containing alcohol solution is placed, within a Ziploc bag, onto the sole of a foot of a patient and illuminated for 30-60 minutes with an ultraviolet (UV) light. Reports on two patients clearly confirmed that Water Gas charged alcohol was more effective than using un-activated alcohol. Moreover, I can use microscopic examination of stored neutral red dye containing alcohol solutions; to determine whether residual, marked light induced motion of un-dissolved material has been fully retained during the storage period.

While solutions containing alcohol are useful for externally enhancing the body's ACE pathway, ideally one could simply ingest inexpensive liquids and foods with ACE promoting activity. The ideal liquid would be water. The planned study was, therefore, to test various water samples for possible intrinsic activity in being able to dissolve neutral red dye in a more dynamic, linear manner with subsequent motions of some of the remaining un-dissolved neutral red dye particles. Several commercially available bottled waters were included in this initial testing including one which claimed to be “electron energized” with negative ORP levels, and another to be “hydrogen rich, twin-vortexed” Neither of the first two water samples promoted a linear dissolving pattern of neutral red dye or enabled persisting movements of un-dissolved dye particles. With some water samples, the mineral concentration appeared to be too high for the neutral red dye to dissolve, e.g. magnesium chloride rich water from the Great Salt Lake, or Quinton plasma rich ocean water. Bubbling Water Gas through ordinary tap water slightly enhance its ability to interact with neutral red dye, but not to an impressive extent.

I then heard of the Bheau View Ranch in San Marcos, Calif. in which the water from a deep well was said to have human health promoting properties. I visited the Ranch and interviewed several individuals who had been consuming the water. One patient had recovered from chronic fatigue syndrome; another patient had regained control of his obesity, hypertension and diabetes (metabolic syndrome). Description of improvements in other patients with a variety of chronic illnesses, including Parkinson's and Alzheimer's diseases, appeared credible. The water from the Ranch was also being used to rehabilitate horses with apparent success, including some horses still present on the Ranch. Finally, I observed luscious algae growth in a very clear pond of the water and many flourishing plants in various locations on the well-watered Ranch. Encouraged by these observations, I collected some water for assessment using the NR Kinetic Assay. Although, not nearly as pronounced as alcohol, it was clear that water from the Ranch was effective in promoting the linear dissolving pattern of neutral red dye particles and also in sustaining light induced motion of un-dissolved neutral red particles, when placed into a well of a tissue culture dish. Moreover, the neutral red containing solution placed in one well of a tissue culture dish was clearly able to induce motion of neutral red dye particles in ordinary tap water and in distilled water in adjacent wells. These observations have validated the utility of the NR Kinetic Assay. The ready availability of the Ranch water is also providing a “Gold Standard” on which to compare other energized waters in agricultural, animal and human studies. For example, although not as marked as the Ranch water, I have discerned NR Kinetic Activity in a commercial water said to be “supercharged with quantum energy.” Had I not had the experience of observing neutral red in alcohol and then in the Ranch water, I might well have discarded the activity in the “supercharged” water.

Understanding the ACE pathway and how it can be enhanced using liquids, foods and other modalities, is providing fresh insights into a new form of energy based medicine and agricultural practices. The described NR Kinetic Assay and any similar, comparable assay method will be of enormous value in moving forward with the science of the ACE pathway. The invention described herein, is intended to cover all related modes of assessing the ability of an energized liquid to transmit energy into regular water; in such a manner that it positively enhances the linear dissolving patterns of dyes, including neutral red, or other chemicals; and/or is able to impart significant motion to un-dissolved neutral red particles, or other suitable particles, which would ordinarily be relatively motionless in the regular water. The naturally energized Ranch water is also of immediate value outside of his current health promoting activity. Included in these new applications are its use as a cosmetic, with added energized alcohol, to improve skin appearance and as a direct method of activating the ACE pathway in skin wound repair and overcoming various skin infections.

As these experiments proceeded, situations were encountered in which water placed in vicinity of a patient, clearly acquired enhanced neutral red dye dissolving activity was observed. The NR Kinetic Assay, therefore, has the potential as a monitoring system for a non-conventional form of energy.

The principles, preferred embodiments and modes of operation of the present invention, intended to be protected herein, is not to be construed as limited to the particular method of assaying for the energetic activity of liquids, which I have linked to activation of the ACE pathway The patent application is not restricted to the use of neutral red as the indicator dye, since other dyes can and have been used in similar experiments. Several dyes have generally shown more rapid and more linear dissolving patterns in the energized water. Neutral red has the advantage, however, of potentially being used in therapeutic endeavors aimed at activating the ACE pathway. ACE pigments, collected from a patient's saliva or perspiration, are also anticipated as being more active in energized, compared with regular water. The term “energized” is also not meant to be exclusive of other relevant terms, which I or other investigators may use in furthering the research described in this application. Additional modifications of the basic tenets disclosed in the present patent application will readily occur to those skilled in the art and especially upon practicing the currently described methods. Therefore, many variations and changes may be made without departing from the scope and spirit of the invention as encompassed by the appended claims.

1. A method for determining the presence of and quantitatively assessing the level of an intrinsic energy property of water; comprising adding a small number of neutral red dye particles to the water and microscopically observing the dye particles, with the understanding that dye particles added to non-energized water will undergo a relatively slow rate of dissolving into soluble neutral red dye, which forms slowly expanding and concentric areas of dye around essentially stationary particles; whereas when a similar number of neutral red dye particles is added to energized water, the particles will dissolve more rapidly and the dissolving dye will form into single linear streams, commonly from moving particles; in addition, only in the energized water, will remaining un-dissolved portions of the original neutral red particles, show persisting movements, including to-and-fro motions, within the light illuminated neutral red dye in water solution.

2. An assay, using the method described in claim 1, to detect the ability of a procedure and/or an energy force, to impart an intrinsic energy property into water, comprising testing samples of water, which have or have not been treated with a procedure and/or exposed to a particular energy force, for the interaction of the water with added particles of neutral red dye.

3. An assay, using the method described in claim 1, to detect the presence of a energy force within an environment, including possibly emanating from an individual; the energy force being detected is that which is able to impart an intrinsic kinetic energy property into water; the testing comprising placing a sample of ordinary water in the environment to be tested, adding a few particles of neutral red dye and observing whether the dye particles show greater kinetic activity, when compared to a similar aliquot of water with added neutral red dye, which has been placed in an ordinary control environment.

4. The method of claim 1, to determine the stability of an intrinsic energy property of water by assessing over time and/or on water samples maintained under different storage conditions, for the interaction of the water samples with added particles of neutral red dye.

5. The method of claim 1, in which an intrinsic energy property of a liquid other than water is being quantitatively assessed, including alcohol containing solutions and beverages, homeopathic and herbal preparations, and bodily fluids.

6. The method of claim 1, in which an intrinsic energy property of water is being quantitatively assessed using dyes or other chemical compounds, which show comparable activity as seen with neutral red dye.

7. The method of claim 1, in which an intrinsic energy property of a neutral red dye containing fluid, which is intended to be used clinically in the activation of the alternative cellular energy (ACE) pathway, is being assessed.
